(19) United States
(12) Patent Application Publication
(10) Pub. No.: US 2010/0189786 A1
(43) Pub. Date: Jul. 29, 2010

(54) CRYSTALLINE TOLTERODINE TARTARATE AND A PHARMACEUTICAL COMPOSITION CONTAINING THE SAME

(76) Inventors: Martin Svoboda, Lazne Bohdanec (CZ); Josef Jampilek, Mlada Boleslav (CZ); Martina Kacirkova, Roztoky (CZ); Vaclav Tomasek, Praha (CZ); Mikulas Lehocky, Praha (CZ); Ludmila Hejtmankova, Prelouc (CZ); Vaclav Vosatka, Praha (CZ)

Correspondence Address: Pearl Cohen Zedek Latzer, LLP, 1500 Broadway, 12th Floor, New York, NY, 10036, US

(21) Appl. No.: 12/376,947

(22) PCT Filed: Aug. 9, 2007
(86) PCT No.: PCT/CZ07/00078

§ 371 Date: Feb. 9, 2009

(30) Foreign Application Priority Data
Aug. 9, 2006 (CZ) PV 2006-506
Feb. 26, 2007 (CZ) PV 2007-160

(51) Int. Cl. A61K 9/28; A61K 31/138; A61P 13/00

(52) U.S. Cl. 424/474; 514/654

(57) ABSTRACT

A crystalline salt of 2-[(1R)-3-[bis(1-methylethyl)amino]-1-phenylpropyl]-4-methyl-phenol with (2R,3R)-2,3-dihydroxybutanedioic acid, known under the name R-tolterodine tartarate, wherein: a) at least 90% of all crystals are present in a size smaller than 30 μm, b) at least 40% of crystalline matter are smaller than 250 μm, c) the maximum size of crystals does not exceed 800 μm, d) the salt contains less than 0.1 weight % of the undesirable enantiomer S-tolterodine tartarate, e) analytical test for sulfate ashes (Pharm. Eur.) provides a value lower than 0.1%. The method of its preparation involves at least one crystallization from water. A pharmaceutical composition containing tolterodine or its pharmaceutically acceptable salts further contains a filler, a disintegrant and a lubricant, said composition being free of ions of alkaline earth metals.

### TECHNICAL FIELD

The invention concerns the salt of 2-[(1R)-3-[bis(1-methylethyl)amino]-1-phenylpropyl]-4-methyl-phenol with (2R,3R)-2,3-dihydroxybutanedioic acid, a known urine spasmolytic, in an advantageous crystalline form for preparation of a pharmaceutical composition. The invention also concerns a pharmaceutical composition containing tolterodine with exceptionally good stability.

### BACKGROUND ART

Tolterodine is a known antagonist of cholinergic receptors, which is used for treatment of unstable urinary bladder, which in turn is associated with urine incontinency.

2-[(1R)-3-[bis(1-methylethyl)amino]-1-phenylpropyl]-4-methyl-phenol, known under the name tolterodine, alternatively R-tolterodine, is used in a drug composition in the form of its salt with (2R,3R)-2,3-dihydroxybutanedioic acid of formula I

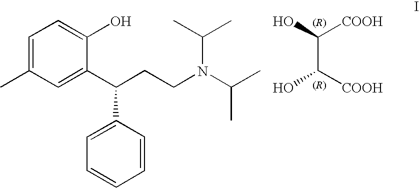

Tolterodine is usually used in the form of salts. The most frequently used is the salt with (2R,3R)-2,3-dihydroxybutanedioic acid (1:1), known also under the name L-tartaric acid.

Tolterodine, a method of its synthesis and its pharmacological effects were first described in patent EP 325 571. The final step of the preparation of R-tolterodine tartarate is described in Example 9 of the mentioned document. Racemic tolterodine is dissolved in ethanol. An alcoholic solution of tartaric acid is added to the solution. The mixture is heated for several minutes and then cooled down, which causes crystallization. In order to obtain the pure substance, the product is again recrystallized from ethanol.

Purity of thus obtained R-tolterodine tartarate is usually not quite satisfactory and the crystallization has to be repeated several times. As the examples below show, even after three recrystallization, the content of undesirable S-tolterodine is still 0.2%. In spite of the fact that the content of organic impurities was satisfactory, the problem seemed to be with inorganic impurities, as follows from the result determination of sulfate ash in the amount 0.34%. These results were borderline satisfactory and did not correspond to ever stricter requirements for pharmaceutically active ingredients. With repeated crystallizations, concentrations of impurities decrease only very slowly at the expense of increasing losses. The question, therefore, was whether the obtained purities are satisfactory for the pharmaceutical compositions or not.

The problem was stability of the pharmaceutical composition. Application of the commonly manufactured pharmaceutical composition mentioned in the cited EP 325 571 did not provide a sufficiently stable product; in the course of time, degradation products were found in the composition in higher concentrations than those tolerable.

The composition described in EP 325 571 has the following composition:

| substance | Amount [mg] |
|---|---|
| Tolterodine | 2 |
| Microcrystalline cellulose | 57 |
| Ca hydrogen phosphate | 15 |
| Sodium carboxymethylstarch | 5 |
| Colloidal silica | 0.25 |
| Mg stearate | 0.75 |
| Total | 80 |

According to the cited patent document, the composition was compressed to tablets, which could but did not have to be further coated.

The composition registered and sold in the Czech Republic corresponds, at least qualitatively, to the above mentioned description, and the version with coating has been selected. The coating is constituted by hydroxypropylmethylcellulose, microcrystalline cellulose, stearic acid and titanium dioxide (data are known only qualitatively). The registered product is wrapped in blisters made of polyvinylchloride, which is coated with a layer of polyvinylidenechloride (PVC/PVDC).

Stability tests showed (Example 1) that while the composition is borderline satisfactory with respect to the stability requirement, the total increase of content of impurities is very high and in the case of improper storing, purity could decrease so much that the product would become unusable.

The problem, therefore, was to prepare a composition that would be absolutely acceptable with respect to stability.

Another problem is with ensuring that the composition containing the product prepared in this way achieves the required release rate of the active substance, which is in turn directly associated with the pharmacological effectiveness of the product. A usual strategy is decreasing the size of crystals. The above described crystallization yields large crystals, which do not allow sufficiently fast release of the active substance and are unsuitable for use. Grinding of thus obtained crystals is difficult. The ground particles are significantly adhesive and tend to stick to the machinery or to form aggregates.

The present invention solves the mentioned problems.

### DISCLOSURE OF INVENTION

The subject matter of the invention consists in a crystalline salt of 2-[(1R)-3-[bis(1-methylethyl)amino]-1-phenylpropyl]-4-methyl-phenol with (2R,3R)-2,3-dihydroxybutanedioic acid, known under the name R-tolterodine tartarate, having the following parameters:
a) at least 90% of all the crystals are present in a size smaller than 30 μm,
b) at least 40% of crystalline matter are smaller than 250 μm,
c) maximum size of crystals does not exceed 800 μm,
   and the purity of which can be further characterized in the following way:
d) it contains less than 0.1 weight % of the undesirable enantiomer S-tolterodine tartarate,
e) analytical test for sulfate ashes (Pharm. Eur.) gives a value lower than 0.1%.

The present invention further consists in a pharmaceutical composition containing tolterodine or its pharmaceutically acceptable salts which, apart from the active substance, contains at least one auxiliary substance from the group of fillers, disintegrants or lubricants, none of said auxiliary substances containing ions of alkaline earth metals.

In the following detailed specification, all the mentioned percents are weight percents.

According to the invention, substances based on carbohydrates can be used as fillers, especially cellulose, starch, lactose or other mono-, oligo- or poly-saccharides. According to the invention, the amount of the filler in the composition ranges from 70 to 95%. It is advantageous to use microcrystalline cellulose as the filler.

For the disintegrant system, one can use for example crospovidone, croscarmellose, carboxymethylstarch. The content of the disintegrant in a tablet ranges from 2 to 10%. Sodium carboxymethylstarch has turned out to be a preferable disintegrant.

Sodium stearyl fumarate, stearic acid, hydrogenated castor oil or aluminum stearate can be used as lubricants. According to the invention, the content of lubricant ranges from 0.5 to 4%.

A composition with these parameters has surprisingly turned out to be significantly more stable than the above-mentioned composition known from the prior art. While in the commercially-sold package of the known composition the content of impurities increased from virtually zero to about 0.8% after 6 months of storing at 40° C. and relative humidity of 75%, in the composition according to the invention the content of impurities does not increase by more than 0.1% (weight percents) under the given conditions. The highest content of individual impurity was almost 0.4% in the case of the known composition; it did not increase by more than 0.05% in the composition according to the invention.

A preferable embodiment of the present invention includes a pharmaceutical composition with said crystalline salt.

As turned out, said composition, using the standard test of active-substance release according to Pharm. Eur. with the paddle method at 50 rpm in the 0.1M HCl medium, at least 40 weight % of the total content of the active substance is dissolved within 5 minutes and at least 60% in a phosphate buffer pH 6.8 under the same conditions.

The composition according to the invention can be prepared via various methods. The methods differ especially with respect to processing of the mixture designed for compression. Because of problems with stability of the preparative, it is better to find a mixture that can be directly tabletted, i.e. compressed without any previous processing. Finding such a mixture leads to further improvement of stability.

Accordingly, the most advantageous solution according to the invention is the one that is produced via direct compression and preferably contains sodium carboxymethylstarch as the disintegrant and sodium stearyl fumarate as the lubricant. Most preferably, the composition is compressed to tablets and furnished with a coating.

The present invention also includes a procedure how to obtain said newly discovered form of the substance. The procedure involves the hitherto undescribed process of crystallization from water.

The procedure preferably comprises suspending crude R-tolterodine tartarate in water, heating the suspension to boil, and maintaining at this temperature until dissolution, followed by crystallization by cooling down of the solution. It is advantageous to use weight ration of R-tolterodine tartarate to water from 1:5 to 1:20, preferably from 1:7 up to 1:15.

The procedure can be performed in such a way that the racemic salt of tolterodine hydrogenbromide is converted, by means of a base, to tolterodine, which is subsequently converted to tartarate by reaction with tartaric acid in a solution of a C1 to C3 alcohol and the corresponding diastereoisomer is crystallized and, after optional crystallization of the product from ethanol, the final crystallization is performed from water.

### DETAILED DESCRIPTION OF THE INVENTION

The individual parameters of the substance R-tolterodine tartarate such as described, according to the problems which they solve:

Problem of Stability of the Pharmaceutical Composition

The known pharmaceutical composition with the active ingredient produced by the know procedure, both according to patent document EP 325 571, did not provide a satisfactory substance. The composition was not stable and the active substance was not released in a sufficient rate.

Detailed testing of the product stability revealed that problems arise due to magnesium or calcium ions, which cause decomposition. For that reason, it was necessary to avoid these ions in the adjuvants. These ions are present in commonly used lubricants. Therefore, those lubricants were used for the composition that are selected from the group of substances including sodium stearyl fumarate, stearic acid, hydrogenated castor oil and aluminum stearate, or their combinations. This has ensured satisfactory short-term stabilities.

Considerations concerning a long-term stable formulation involved the apparent requirement to remove even trace amounts of these elements, which were determined in the first approximation via measuring the sulfate ashes. As far as stability is concerned, this impurity, therefore, has turned out to be very important. Considering the negative influence of these elements, it was clear that a substance that should be stable for a long time has to show low content of sulfate ash. Values below 0.3% have turned out to be satisfactory.

Problem of the Release Rate

R-tolterodine tartarate is poorly soluble in water and, therefore, problems with the rate of its release into water media could be anticipated. The release rate plays an important role in bioavailability of the product. A common strategy how to improve the availability is grinding of the product. However, in the case of R-tolterodine tartarate, it was very difficult to grind the particles. It has turned out that the particles contained either too large crystals or too high content of powder particles, which made it difficult to handle the material.

Basically, it was possible to grind the material to very small particles and thus to obtain for example up to 90% of all particles smaller than 30 μm. However, very small particles created aggregates, which probably did not disintegrate in the water medium, and the release rate problem thus was not solved completely. For a successful formulation, the condition of weight ratios has turned to be important; i.e. it is necessary that at least 40% of all particles pass through the 250 μm sieve. The last condition included ruling out the incidence of isolated large crystals. In spite the fact that their number is quite negligible, they can represent a significant share by weight. Therefore, no crystal should exceed 800 μm in size.

Problem of Undesirable S-Tolterodine

Optical purity is an obvious important requirement for pharmaceutical substances. In the case of R-tolterodine tartarate, the content of the opposite enantiomer after three crystallizations from ethanol is about 0.2%. This value has seemed to be satisfactory so far. However, the requirements concerning purity of the products usable in pharmaceutical compositions are getting ever stricter. Substances with the S-tolterodine content lower than 0.1% are better suited to meet these new requirements.

The Pharmaceutical Composition

In the preparation of the pharmaceutical composition the question was how the active pharmaceutical ingredient of new parameters would influence the properties of the pharmaceutical composition. The composition according to the invention was prepared in the form of coated tablets. It has turned out that it displays excellent release profiles of active substance. Especially in the neutral environment of the phosphate buffer (pH 6.8), a substantial portion of the active substance was released already during the first 5 minutes.

For combining the good release rate of the active substance with the stability of the pharmaceutical composition, the composition containing the active substance in an amount of from 1 to 2.5%, from 70 to 95% of a filler, from 2 to 10% of a disintegrant and from 0.5 to 4% of a lubricant has turned out to be advantageous, the lubricant being selected from the group of substances including sodium stearyl fumarate, stearic acid, hydrogenated castor oil and aluminum stearate, or their combinations.

Procedure for Preparing the Active Ingredient

R-tolterodine tartarate itself belongs to pharmaceutical ingredients that are poorly soluble in cold water, which results in the above-mentioned problems with release of the active-substance from the pharmaceutical composition into aqueous media. This is probably why crystallization from ethanol is described in the literature.

Surprisingly, it has turned out that crystallization from water yields an active substance that meets the above-discussed parameters of the product.

### BRIEF DESCRIPTION OF DRAWINGS

The drawings represent results of measurement of the particle size of the product prepared according to Example 2 by a microscopic method.

FIG. 1 represents batch I, where 91.95% of all particles are smaller than 30 μm; 40.9% of weight of the material would pass through the 250 μm sieve and the maximum crystal size is 623 μm.

FIG. 2 represents batch II, where 94.73% of particles are smaller than 30 μm; 47.2% of weight of the material would pass through the 250 μm sieve and the maximum crystal size is 439 μm.

FIG. 3 represents batch III, where 89.29% of particles are smaller than 30 μm; 41.3% of weight of the material would pass through the 250 μm sieve and the maximum crystal size is 702 μm.

### EXAMPLES

The invention is illustrated in more detail in the following examples.

### Example 1

### The Pharmaceutical Composition According to EP 325 571

The same qualitative composition of the formulation as for the commercially sold product was used for preparation of laboratory batches.

Technology of direct compression was used for processing the formulation.

Commercially sold product 1 and 2 mg

Formulation—Coated Tablets

Composition

| core | coating |
|---|---|
| microcrystalline cellulose | hypromellose |
| CaHPO₄•2H₂O | microcrystalline cellulose |
| explotab | stearic acid |
| magnesium stearate | titanium oxide |
| colloidal silicon oxide | |

TABLE NO. 1
Stability evaluation of the prepared product (labeled batch 020104) and of the
commercially manufactured product

| | | purity (%) | | | | | | | | |
|---|---|---|---|---|---|---|---|---|---|---|
| | | Individ. impur. max. 0.2%/sum of impur. max. 0.5% | | | | | | | | |
| | | | 3 months | | | | 6 months | | | |
| | | | | | PVC/PVDC/ | | | | PVC/PVDC/ | |
| | | | PVC/Al | | Al | | PVC/Al | | Al | |
| | batch | input | blister | | blister | | blister | | blister | |
| Compn. | 02 01 04 | <0.05 | <0.05 | <0.05 | <0.05 | <0.05 | <0.05 | <0.05 | <0.05 | <0.05 |
| according | 25° C./60% RH | | | | | | | | | |
| to original | 02 01 04 | | 0.07 | 0.12 | 0.05 | 0.05 | 0.08 | 0.08 | 0.07 | 0.07 |
| | 30° C./65% RH | | | | | | | | | |
| | 02 01 04 | | 0.26 | 0.56 | 0.22 | 0.36 | 0.56 | 0.92 | 0.49 | 0.83 |
| | 40° C./75% RH | | | | | | | | | |
| | Commercial | — | | | 0.28 | 0.67 | | | 0.39 | 0.86 |
| | product | | | | | | | | | |
| | 1 mg | | | | | | | | | |
| | 40° C./75% RH | | | | | | | | | |

Stability evaluation was performed in two packagings, PVC and PVC/PVDC.

For each type of packaging, the left column presents the content of the highest-amount impurity and the right column presents the sum of all impurities.

After 3 months at 40° C. and 75% relative humidity, the sum of impurities increased to 0.56% in the PVC packaging and to 0.36% in the PVC/PVDC packaging.

After 6 months at 40° C. and 75% relative humidity, the sum of impurities increased to 0.92% in the PVC packaging and to 0.83% in the PVC/PVDC packaging, similarly as in the original commercially sold product. See Table No. 1.

### Example 2

### Reference

A new composition without calcium hydrogenphosphate dihydrate was proposed for the pilot batches.

| Core | coating |
|---|---|
| microcrystalline cellulose | hypromellose |
| explotab | stearic acid |
| magnesium stearate | titanium dioxide |
| colloidal silicon oxide | microcrystalline cellulose |

The product is unsatisfactory with respect to the content of impurities after 3 months at 40° C./75% r.h., after 6 months at 30° C./65% r.h. and after 9 months at the normal conditions, i.e. 25° C./60% r.h. See the tables of stability evaluation.

Stability Evaluation

Batch Number: 0413 09 04 PVC, Al blister, 40° C./75% r.h.

| time | impurity | impurity | impurity | impurity | | Sum of | Optical purity |
|---|---|---|---|---|---|---|---|
| [month] | (RRT 0.85) | (RRT 1.08) | (RRT 1.88) | (RRT 1.90) | Other impurities | impurities | (S) enantiomer |
| 0 | <0.05% | <0.05% | <0.05% | <0.05% | <0.05% | <0.05% | 0.2% |
| 3 | 0.38% | — | 0.09% | 0.69% | <0.05% | 1.16% | 0.32% |
| 6 | 0.48% | 0.05% | 0.20% | 1.12% | <0.05% | 1.85% | 0.69% |

Batch Number: 0413 09 04 PVC, Al blister, 30° C./65% r.h.

| time | impurity | impurity | impurity | impurity | | Sum of | Optical purity |
|---|---|---|---|---|---|---|---|
| [month] | (RRT 0.85) | (RRT 1.08) | (RRT 1.88) | (RRT 1.90) | Other impurities | impurities | (S) enantiomer |
| 0 | <0.05% | <0.05% | <0.05% | <0.05% | <0.05% | <0.05% | 0.2% |
| 3 | 0.22% | — | 0.05% | 0.38% | <0.05% | 0.65% | 0.2% |
| 8 | 0.31% | 0.07% | 0.12% | 0.74% | <0.05% | 1.24% | 0.34% |

Batch Number: 0413 09 04 PVC, Al blister, 25° C./60% r.h.

| time | impurity | impurity | impurity | impurity | | Sum of | Optical purity |
|---|---|---|---|---|---|---|---|
| [month] | (RRT 0.85) | (RRT 1.08) | (RRT 1.88) | (RRT 1.90) | Other impurities | impurities | (S) enantiomer |
| 0 | <0.05% | <0.05% | <0.05% | <0.05% | <0.05% | <0.05% | 0.2% |
| 3 | 0.10% | — | 0.13% | — | <0.05% | 0.23% | 0.23% |
| 6 | 0.14% | 0.07% | — | 0.25% | <0.05% | 0.46% | 0.32% |
| 9 | 0.19% | 0.08% | 0.08% (1.81) | 0.42% | <0.05% | 0.77% | 0.35% |

Batch Number: 0513 09 04 PVC, Al blister, 40° C./75% r.h.

| time | impurity | impurity | impurity | impurity | | Sum of | Optical purity |
|---|---|---|---|---|---|---|---|
| [month] | (RRT 0.85) | (RRT 1.08) | (RRT 1.88) | (RRT 1.90) | Other impurities | impurities | (S) enantiomer |
| 0 | <0.05% | — | <0.05% | <0.05% | <0.05% | <0.05% | 0.2% |
| 3 | 0.39% | — | 0.08% | 0.71% | <0.05% | 1.18% | 0.31% |
| 6 | 0.45% | 0.05% | 0.19% | 1.08% | <0.05% | 1.77% | 0.71% |

Batch Number: 0513 09 04 PVC, Al blister, 30° C./65% r.h.

| time | impurity | impurity | impurity | impurity | | Sum of | Optical purity |
|---|---|---|---|---|---|---|---|
| [month] | (RRT 0.85) | (RRT 1.08) | (RRT 1.88) | (RRT 1.90) | Other impurities | impurities | (S) enantiomer |
| 0 | <0.05% | | <0.05% | <0.05% | <0.05% | <0.05% | 0.2% |
| 3 | 0.21% | | <0.05% | 0.37% | <0.05% | 0.58% | 0.22% |
| 8 | 0.31% | 0.08% | 0.13% | 0.70% | <0.05% | 1.22% | 0.34% |

Batch Number: 0513 09 04 PVC, Al blister, 25° C./60% r.h.

| time | impurity | impurity | impurity | impurity | | Sum of | Optical purity |
|---|---|---|---|---|---|---|---|
| [month] | (RRT 0.85) | (RRT 1.08) | (RRT 1.88) | (RRT 1.90) | Other impurities | impurities | (S) enantiomer |
| 0 | <0.05% | <0.05% | <0.05% | <0.05% | <0.05% | <0.05% | 0.2% |
| 3 | 0.09% | — | 0.12% | — | <0.05% | 0.21% | 0.25% |
| 6 | 0.14% | 0.08% | 0.23% | — | <0.05% | 0.45% | 0.37% |
| 9 | 0.18% | 0.08% | 0.07% (1.81) | 0.39% | <0.05% | 0.72% | 0.34% |

### Example 3

Stress Tests:

In the following tests, an increase of impurities was measured for binary mixtures of the active agent and lubricant in ratio 1:1.5 (weight) in humid air at the temperature of 80° C., for 6 hours.

Stress Tests

Comparison - stress burdens of binary mixtures 80° C./6 hours/wet warmth

| | tolterodine tartarate | tolterodine tartarate | tolterodine tartarate | tolterodine tartarate |
|---|---|---|---|---|
| Impurities | b. 031111 | b. 031111 | b. 031111 | b. 031111 |
| individually | Magnesium stearate | Aluminum stearate | Hydrogen, castor oil | Stearic acid |
| impurity RRT 0.86 | 1.31% | 0.07% | <0.05% | 0.05% |
| impurity RRT 1.07 | 0.46% | 0.13% | 0.11% | 0.10% |
| impurity RRT 1.10 | <0.05% | <0.05% | 0.06% | <0.05% |
| impurity RRT 1.81 | 0.51% | <0.05% | 0.04% | <0.05% |
| impurity RRT 1.90 | 2.95% | 0.09% | <0.05% | 0.05% |

It has turned out that the increase of impurities in the presence of magnesium stearate is several times higher than in the case of other tested lubricants.

### Example 4

A new composition without calcium hydrogenphosphate dihydrate and magnesium stearate. Magnesium stearate was replaced by sodium stearyl fumarate.

After 6 months at 40° C./75% r.h. as well as at 25° C./60% r.h., there is no increase of the content of impurities. See the tables of stability evaluation.

Composition for 1 and 2 mg strengths of Tolterodine tartarate.

| | Amount [mg] | Amount [mg] |
|---|---|---|
| core | | |
| Tolterodine tartarate | 1.00 | 2.00 |
| Microcrystalline cellulose | 73.00 | 146.00 |
| Sodium salt of | 3.50 | 7.00 |
| carboxymethylstarch | | |
| Colloidal silicon oxide | 1.00 | 2.00 |
| Sodium stearyl fumarate | 1.50 | 3.00 |
| Total | 80.00 | 160.00 |
| coating | | |
| Hypromellose | 1.75 | 3.50 |
| Macrogol 6000 | 0.30 | 0.60 |
| Titanium dioxide | 0.15 | 0.40 |
| Iron monoxide | 0.05 | — |
| Talc | 0.25 | 0.50 |
| Total | 2.50 | 5.00 |

Stability Evaluation

TOLTERODINE 1 tbl. flm., Batch Number: 10 01 06, Pilot batch
Packaging: PVC/PVDC, Al, Storing conditions: 40° C./75% R.H.
Purity test

| | Known | Unknown | Sum of all | Optical purity |
|---|---|---|---|---|
| time | impurities | impurities | impurities | (S) enantiomer |
| [month] | Max. 0.5% | Max. 0.5% | Max. 1.0% | Max. 0.5% |
| 0 | <0.05% | <0.05% | <0.05% | <0.12% |
| 3 | <0.05% | <0.05% | <0.05% | — |
| 6 | <0.05% | <0.05% | <0.05% | |

TOLTERODINE 1 tbl. flm., Batch Number: 11 01 06, Pilot batch
Packaging: PVC/PVDC, Al, Storing conditions: 40° C./75% R.H.
Purity test

| | Known | Unknown | Sum of all | Optical purity |
|---|---|---|---|---|
| time | impurities | impurities | impurities | (S) enantiomer |
| [month] | Max. 0.5% | Max. 0.5% | Max. 1.0% | Max. 0.5% |
| 0 | <0.05% | <0.05% | <0.05% | <0.12% |
| 3 | <0.05% | <0.05% | <0.05% | — |
| 6 | <0.05% | <0.05% | <0.05% | |

TOLTERODINE 1 tbl. flm., Batch Number: 10 01 06, Pilot batch
Packaging: PVC/PVDC, Al, Storing conditions: 25° C./60% R.H.

| | Purity test | | | |
|---|---|---|---|---|
| | Known | Unknown | Sum of all | Optical purity |
| time | impurities | impurities | impurities | (S) enantiomer |
| [month] | NMT 0.5% | NMT 0.5% | NMT 1.0% | NMT 0.5% |
| 0 | <0.05% | <0.05% | <0.05% | <0.12% |
| 3 | <0.05% | <0.05% | <0.05% | — |
| 6 | <0.05% | <0.05% | <0.05% | |
| 9 | | | | |
| 12 | | | | |

TOLTERODINE 1 tbl. flm., Batch Number: 11 01 06, Pilot batch
Packaging: PVC/PVDC, Al, Storing conditions: 25° C./60% R.H.

| | Purity test | | | |
|---|---|---|---|---|
| | Known | Unknown | Sum of all | Optical purity |
| time | impurities | impurities | impurities | (S) enantiomer |
| [month] | NMT 0.5% | NMT 0.5% | NMT 1.0% | NMT 0.5% |
| 0 | <0.05% | <0.05% | <0.05% | <0.12% |
| 3 | <0.05% | <0.05% | <0.05% | — |
| 6 | <0.05% | <0.05% | <0.05% | |
| 9 | | | | |
| 12 | | | | |

### Example 5

Similarly as the composition in Example 4, the following composition also showed very good stability properties:

| core | Amount [mg] |
|---|---|
| Tolterodine tartarate | 1.00 |
| Spray-dried lactose | 69.00 |
| Crospovidone | 7.00 |
| Colloidal silicon oxide | 1.00 |
| Sodium stearyl fumarate | 2.00 |
| Total | 80.00 |

The mixture was prepared in a homogenizer of the rotating-pinion type. A mixture of the first four components was stirred for 15 minutes after sifting, followed by addition of sodium stearyl finnarate, and the mixture was homogenized for additional 5 minutes. Then, the mixture was transported, using a belt conveyor, to a tablet forming machine, where it was compressed.

### Example 6

### Testing of Crystallization

R-tolterodine tartarate was prepared according to the scheme:

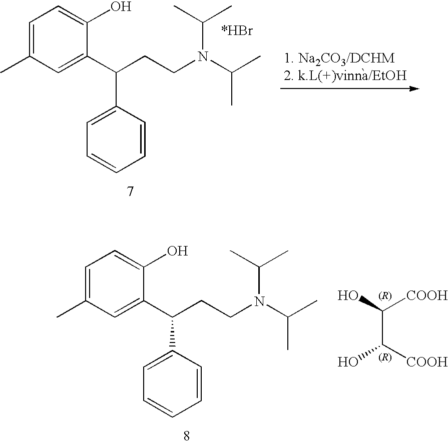

Apparatus: Glass Separator 80 l Equipped With a Stirrer

Procedure: Tolterodine hydrogenbromide 7 was stirred in a mixture of 5% Na₂CO₃ and dichloromethane until the product dissolved, about 1 hour. The organic phase was separated and filtered. The aqueous phase was extracted twice more with dichloromethane. The combined organic phases were extracted with water and concentrated in a rotary vacuum evaporator. The obtained tolterodine base was dissolved in ethanol and a solution of L(+)tartaric acid in ethanol was added. After adding a seed, the solution was left without stirring at room temperature until the next day. After filtration, the crude product was crystallized from ethanol, Batch I three times, Batches II and III twice, about 50 liters of the solvent for 1 kg of R(+)tolterodine tartarate. The fourth crystallization of Batch I and the third crystallization of Batches II and Batch III were performed from water, about 10 liters of water for 1 kg of R(+)tolterodine tartarate.

| | |
|---|---|
| Theoretical yield: | I 2.11 II 4.21 III 4.45 kg |

Actual Yield:

| | | | | | |
|---|---|---|---|---|---|
| Batch I: | 1.0 kg, i.e. 47.4% | HPLC | 99.63% | S(−)tolter- | 13.4% |
| Batch II: | 2.4 kg, i.e. 57.0% | quality | 99.70% | odine | |
| Batch III: | 2.1 kg, i.e. 47.2% | | 99.84% | | |

Ist Crystallization:

Yield:

| | | | | | |
|---|---|---|---|---|---|
| Batch I: | 0.87 kg, i.e. 80.0% | HPLC | 99.77% | S(−)tolter- | 3.3% |
| Batch II: | 1.74 kg, i.e. 72.5% | quality | 99.70% | odine | 3.7% |
| Batch III: | 1.60 kg, i.e. 76.2% | | 99.27% | | 3.2% |

IInd crystallization:

Yield:

| | | | | | |
|---|---|---|---|---|---|
| Batch I: | 0.62 kg, i.e. 77.5% | HPLC | 99.85% | S(−)tolter- | 0.70% |
| Batch II: | 1.50 kg, i.e. 86.2% | quality | not an- | odine | 0.76% |
| | | | alyzed | | |
| Batch III: | 1.33 kg, i.e. 83.1% | | 99.89% | | 0.85% |

After two crystallizations from ethanol, the content of S-Tolterodine was still unsatisfactory.

IIIrd crystallization:

Yield:

| | | | | | |
|---|---|---|---|---|---|
| Batch I: | 0.53 kg, i.e. 85.5% | HPLC | 99.73% | S(−)tolter- | 0.19% |
| Batch II: | 1.26 kg, i.e. 84.0% | quality | 99.95% | odine | <0.07% |
| Batch III: | 1.13 kg, i.e. 85.0% | | 100.0% | | <0.07% |

After crystallization from water, the content of S-tolterodine decreased to an immeasurable value (<0.07%) for both batches. In the case of crystallization from ethanol, it oscillated on the borderline of acceptability 0.2%. After three crystallizations from ethanol, the content of sulfate ash was still not satisfactory in Batch I, it was 0.34%. Therefore, one more crystallization was performed.

| | | | | | |
|---|---|---|---|---|---|
| Batch I: | 0.40 kg, i.e. 76.0% | HPLC | 99.81% | S(−)tolter- | <0.07% |
| | | quality | | odine | |

After the crystallization, the content of sulfate ash was 0.03%. The content of S-tolterodine also dropped to the expected value.

For all the batches, measurement of particle size was performed without any treatment using the microscopic method.

Results:

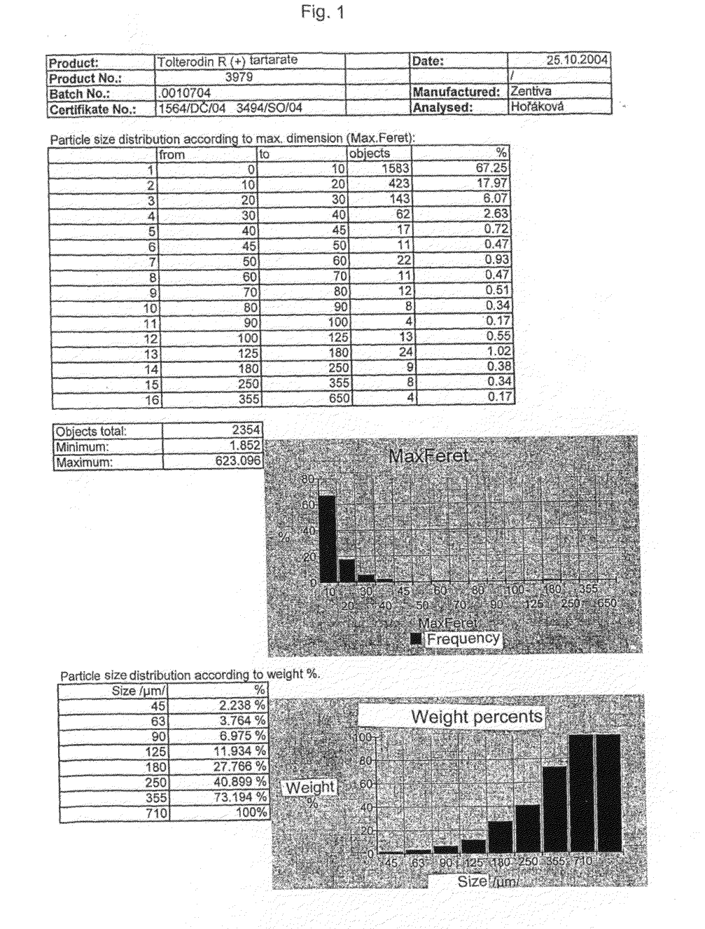

Batch I (FIG. 1): 91.95% of all particles smaller than 30 μm; 40.9% of weight of the material would pass through the 250 μm sieve and the maximal crystal size is 623 μm.

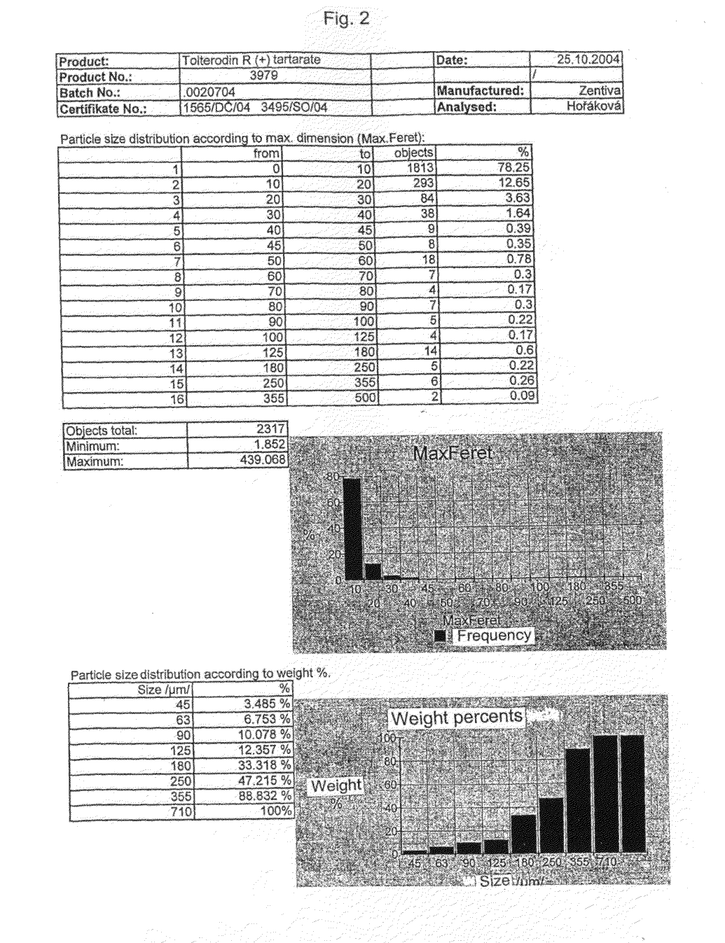

Batch II (FIG. 2): 94.73% of particles smaller than 30 μm; 47.2% of weight of the material would pass through the 250 μm sieve and the maximal crystal size is 439 μm.

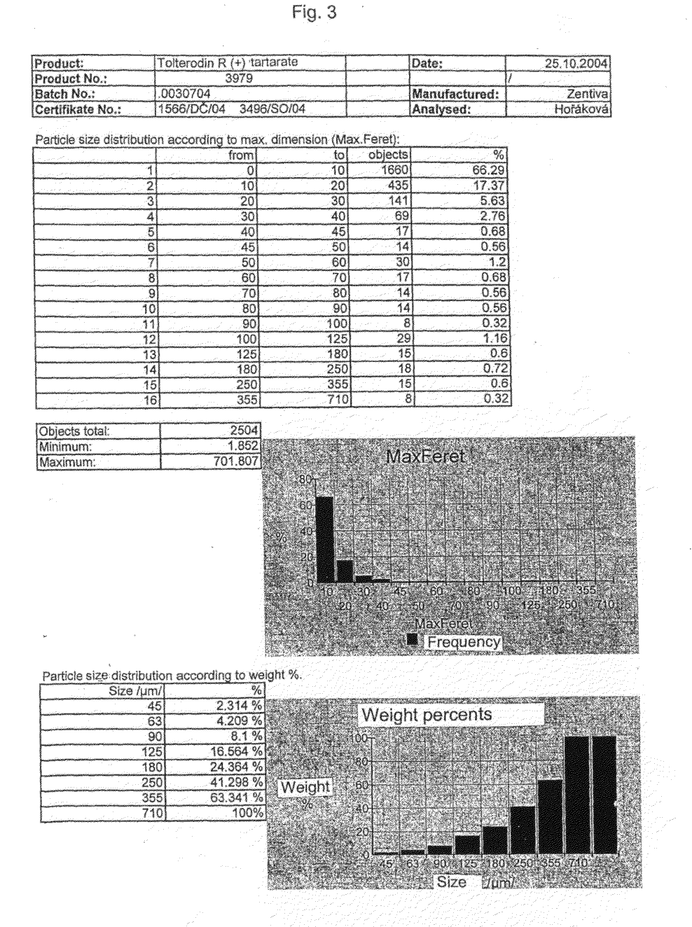

Batch III (FIG. 3): 89.29% of particles smaller than 30 μm; 41.3% of weight of the material would pass through the 250 μm sieve and the maximal crystal size is 702 μm.

### Example 7

The Most Advantageous Method of Preparation of API

Tolterodine hydrobromide 7 in the amount 3.4 kg (8.4 mol) of was stirred in a mixture of 5% Na₂CO₃, which was prepared by dissolving 0.9 kg of sodium carbonate in water and adjusting the volume to 18 liters of the solution and 27 liters of dichloromethane for ca. 60 minutes. The organic phase was separated, the aqueous layer was extracted two more times with 5 liters of dichloromethane. The solvent was distilled out from the combined organic phases. The obtained tolterodine base was dissolved in 30 liters of ethanol and a solution of 1.26 kg (8.4 mol) of L(+)tartaric acid in 65 l of ethanol was added. The solution was stirred for about 2 hours and then placed in a refrigerator at the temperature of about 5-8° C. The next day the precipitated product was filtered. The crystals were washed with cold ethanol and dried. The crude product was first crystallized from ethanol. R-Tolterodine tartarate in the amount of 2.2 kg was dissolved in 110 liters of ethanol under reflux. The solution was left to cool down spontaneously without stirring until the next day. The precipitated crystals were filtered and dried. The final crystallization was performed from water. The product crystallized from ethanol in the amount 1.76 kg was dissolved in 18 liters of water under reflux. The solution was left to cool down spontaneously without stirring until the next day. The precipitated crystals were filtered and dried. 1.5 kg of R-tolterodine tartarate of declared quality were obtained.

### Example 8

R-tolterodine tartarate prepared in Example 3 was used for manufacturing a pharmaceutical composition by direct compression (tabletting without previous processing of the tablet-forming mixture).

Composition for 1 and 2 mg strengths of R-tolterodine tartarate.

| | Amount [mg] | Amount [mg] |
|---|---|---|
| core | | |
| R Tolterodine tartarate | 1.00 | 2.00 |
| Microcrystalline cellulose | 73.00 | 146.00 |
| Sodium carboxymethylstarch | 3.50 | 7.00 |
| Colloidal silicon oxide | 1.00 | 2.00 |
| Sodium stearyl fumarate | 1.50 | 3.00 |
| Total | 80.00 | 160.00 |
| Coating | | |
| Hypromellose | 1.75 | 3.50 |
| Macrogol 6000 | 0.30 | 0.60 |
| Titanium oxide | 0.15 | 0.40 |
| Iron monoxide | 0.05 | — |
| Talc | 0.25 | 0.50 |
| Total | 2.50 | 5.00 |

After 6 months at 40° C./75% relative humidity as well as at 25° C./60% relative humidity, the content of impurities does not increase. See the tables of stability evaluation.

Test of purity Storing conditions: 40° C./75% relative humidity

| | Known | Unknown | Sum of all | Optical purity |
|---|---|---|---|---|
| time | impurities | impurities | impurities | (S)enantiomer |
| [months] | Max. 0.5% | Max. 0.5% | Max. 1.0% | Max. 0.5% |
| 0 | <0.05% | <0.05% | <0.05% | <0.12% |
| 3 | <0.05% | <0.05% | <0.05% | — |
| 6 | <0.05% | <0.05% | <0.05% | |

Release rate of the active substance under conditions defined in Pharm. Eur., measured with the paddle method at 50 rpm:

Release of active substance in 0.1 M HCl

| | % active |
|---|---|
| T[min] | substance |
| 0 | 0 |
| 5 | 56.6 |
| 10 | 81.07 |
| 15 | 89.95 |
| 20 | 94.28 |
| 30 | 98.51 |

It turns out that more than 50% of the active substance is released already in 5 minutes, 90% is released in 15 minutes, and virtually all the active substance is released after 30 minutes.

Release of active substance in phosphate buffer at pH 6.8

| | % active |
|---|---|
| T[min] | substance |
| 0 | 0 |
| 5 | 74.2 |
| 10 | 83.31 |
| 15 | 87.97 |
| 20 | 90.46 |
| 30 | 93.49 |

More than 70% of the active substance is dissolved in 5 minutes and more than 90% in 20 minutes.

1. A crystalline salt of 2-[(1R)-3-[bis(1-methylethyl)amino]-1-phenylpropyl]-4-methyl-phenol with (2R,3R)-2,3-dihydroxybutanedioic acid, known under the name R-tolterodine tartarate, wherein:
a) at least 90% of all crystals are present in a size smaller than 30 μm;
b) at least 40% of crystalline matter are smaller than 250 μm;
c) the maximum size of crystals does not exceed 800 μm;
d) it contains less than 0.1 weight % of the undesirable enantiomer S-tolterodine tartarate; and
e) analytical test for sulfate ashes (Pharm. Eur.) provides a value lower than 0.1%.

2. A method of preparation of R-tolterodine tartarate according to claim 1, wherein the preparation involves at least one crystallization of this substance from water.

3. The method according to claim 2, wherein crude R-tolterodine tartarate is suspended in water, the suspension is heated to boil, and is kept at this temperature until dissolution, followed by crystallization by cooling down the solution.

4. The method according to claim 3, wherein the weight ratio of R-tolterodine tartarate to water is from 1:5 to 1:20.

5. The method according to claim 3, wherein the weight ratio of R-tolterodine tartarate to water is from 1:7 to 1:15.

6. The method according to claim 2, wherein the racemic salt of tolterodine hydrogenbromide is first converted, by action of a base, to tolterodine, which is subsequently converted into the tartarate by reaction with tartaric acid in a solution of a C1 to C3 alcohol and the respective diastereoisomer is crystallized and, after optional crystallization of the product from ethanol, the final crystallization is performed from water.

7. A pharmaceutical composition containing tolterodine tartarate as the active substance, a filler, a disintegrant and a lubricant, said composition being free of ions of alkaline earth metals.

8. The pharmaceutical composition according to claim 7, wherein the composition is free of Mg²⁺ and Ca²⁺ ions.

9. The pharmaceutical composition according to claim 7, wherein the pharmaceutical composition contains, by weight, the active substance in the amount of 1 to 2.5%, 70 to 95% of the filler, 2 to 10% of the disintegrant and 0.5 to 4% of the lubricant.

10. The pharmaceutical composition according to claim 7, wherein the lubricant is selected from the group including sodium stearyl fumarate, stearic acid, hydrogenated castor oil and aluminum stearate, or their combinations.

11. The pharmaceutical composition according to claim 7, wherein the pharmaceutical composition contains crystalline salt of 2-[(1R)-3-[bis(1-methylethyl)amino]-1-phenylpropyl]-4-methyl-phenol with (2R,3R)-2,3-dihydroxybutanedioic acid, known under the name R-tolterodine tartarate, wherein:
f) at least 90% of all crystals are present in a size smaller than 30 μm;
g) at least 40% of crystalline matter are smaller than 250 μm;
h) the maximum size of crystals does not exceed 800 μm;
i) it contains less than 0.1 weight % of the undesirable enantiomer S-tolterodine tartarate; and
j) analytical test for sulfate ashes (Pharm. Eur.) provides a value lower than 0.1%.

12. The pharmaceutical composition according to claim 7, wherein the pharmaceutical composition contains R-tolterodine tartarate that is obtained by a method involving at least one crystallization of this substance from water.

13. The pharmaceutical composition according to claim 11, wherein in the standard test of release of the active substance according to Pharm. Eur. using the paddle method at 50 rpm, at least 40 weight % of the total content of the active substance is dissolved in 0.1M HCl within 5 minutes and at least 60% is dissolved in the phosphate buffer, pH 6.8, under the same conditions.
